(19) United States
(12) Patent Application Publication
(10) Pub. No.: US 2008/0125603 A1
(43) Pub. Date: May 29, 2008

(54) Method For The Production Of Monoalkylated Diamines

(75) Inventors: Werner Hubert Russ, Florsheim-Wicker (DE); Michael Hutchings, Bury (GB); Warren J. Ebenezer, Greater Manchester (GB)

Correspondence Address: CONNOLLY BOVE LODGE & HUTZ, LLP, P O BOX 2207, WILMINGTON, DE, 19899, US

(73) Assignee: DYSTAR TEXTILFARBEN GMBH & CO. DEUTSCHLAND KG, Frankfurt am Main (DE)

(21) Appl. No.: 11/658,817

(22) PCT Filed: Jul. 22, 2005
(86) PCT No.: PCT/EP05/53591

§ 371 Date: Feb. 27, 2007

(30) Foreign Application Priority Data
Jul. 29, 2004 (DE) 10 2004 036 787.6

(51) Int. Cl. C07C 229/00; C07C 211/43

(52) U.S. Cl. 562/433; 564/305

(57) ABSTRACT

Process for preparing alkylated amines

The present invention relates to a process for preparing a compound of the formula (I)

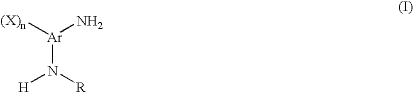

by reacting a compound of the formula (II)

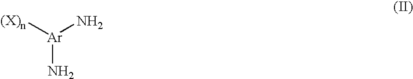

with a compound of the formula (III)

(RO)₂CO (III)

or a compound of the formula (IV)

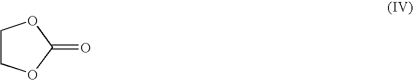

in the presence of a zeolite of the NaY faujasite type, where Ar, X, R and n are each as defined in claim 1.

The present invention relates to the preparation of mono-N-alkylated aromatic diamines, which constitute valuable intermediates, for example for the preparation of dyes.

J. Org. Chem. 2003, 68, 7374-7378 discloses the methylation of aromatic amines with dimethyl carbonate in the presence of a zeolite of the NaY faujasite type.

It has now been found that, surprisingly, this process leads, when employed on aromatic diamines, selectively to the mono-N-alkylation only of one amino group, while the second amino group remains unchanged.

The present invention accordingly relates to a process for preparing a compound of the formula (I)

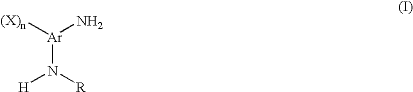

in which
Ar is an aromatic or heteroaromatic ring system;
X is a substituent;
n is from 0 to 6; and
R is an alkyl, hydroxyalkyl or a benzyl group;
   by reacting a compound of the formula (II)

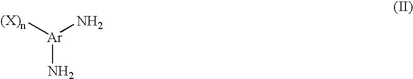

in which Ar, X and n are each as defined above with a compound of the formula (III)

(RO)₂CO (III)

in which R is as defined above or a compound of the formula (IV)

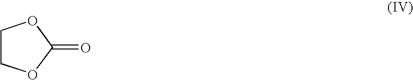

in the presence of a zeolite of the NaY faujasite type.

In a preferred process according to the invention,
Ar is a benzene ring, a fused benzene ring, a pyridine ring or a pyrimidine ring;
X is C₁-C₄-alkyl, —COOR¹, —COR¹, —OR¹, —NHCOR¹, SO₂R², halogen, cyano, nitro, —SO₃M or C₁-C₄-alkyl which is substituted by OR¹, OCOR¹ or by NHCOR¹,
   in which R¹ is hydrogen, C₁-C₄-alkyl or C₁-C₄-alkyl which is substituted by OR¹, OCOR¹ or NHCOR¹;
   R² is C₁-C₄-alkyl, halogen or C₁-C₄-alkyl which is substituted by OH or by OCOR¹; and
   M is hydrogen or an alkali metal such as sodium, potassium or lithium;
n is 0, 1 or 2; and
R is C₁-C₄-alkyl, or hydroxy-C₁-C₄-alkyl;

where the —NH₂ group and the —NHR group may be ortho or meta to one another.

C₁-C₄-Alkyl groups may be straight-chain or branched and are, for example, methyl, ethyl, n-propyl, isopropyl, n-butyl, isobutyl, sec-butyl or tert-butyl. The same applies analogously to hydroxy-C₁-C₄-alkyl. Halogen is, for example, fluorine, chlorine or bromine.

In a preferred process according to the invention,
Ar is a benzene ring;
X is methyl, ethyl, methoxy, ethoxy, —COOH, —COOMe, —COOEt, —NHCOMe, chlorine, cyano or nitro;
n is 0, 1 or 2; and
R is methyl, ethyl or hydroxyethyl.

In a very preferred process according to the invention, the compound of the formula (I) has the formula (Ia)

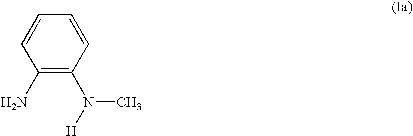

In a further preferred process according to the invention, the catalyst used is a modified zeolite of the NaY faujasite type, and particular preference is given to a modification by addition of a lithium salt cocatalyst, for example lithium bicarbonate or lithium carbonate.

In a further variant of the process according to the invention, it is carried out in a closed reaction vessel under pressure, so that reaction temperatures above the boiling point of the reaction mixture are possible. The reaction mixture may be placed under pressure, for example up to about 20 bar.

In a further variant of the process according to the invention, this heating, necessary in some cases, of the reaction mixture is performed in a microwave oven.

In a further variant of the process according to the invention, it is performed under an inert gas atmosphere, for example under nitrogen or argon, in order to prevent undesired reactions of the reactants or of the product, for example oxidation reactions.

Zeolites of the NaY faujasite type are known and may be purchased commercially.

The compounds of the formula (I) are valuable intermediates, for example, for the preparation of dyes, for instance of reactive dyes, as described in EP 0 256 650 A1 and EP 0 315 045 A1.

### EXAMPLE 1

1.0 g of predried zeolite of the NaY faujasite type was mixed with 1.0 g (9.2 mmol) of ortho-phenylenediamine and 30 ml of dimethyl carbonate (0.31 mol). The mixture was boiled at reflux for 5 hours until chromatographic checking indicated completeness of conversion. The mixture was cooled to room temperature, then the catalyst was filtered off and washed with methanol. The combined filtrates were concentrated by evaporation under reduced pressure and gave rise to 0.84 g of N-methyl-ortho-phenylenediamine in the form of a viscous yellow oil.

¹H NMR and mass spectra of the resulting compound were in full agreement with the expectations. Co-chromatography with a comparative sample obtained from Aldrich likewise showed agreement, and the absence of more highly alkylated compounds.

### EXAMPLES 2 to 8

Analogously to the above example 1, the compounds of the formula (II) specified below may also be converted to the corresponding end products of the formula (I). In the table below, DMC means dimethyl carbonate, DEC means diethyl carbonate and EC means ethylene carbonate (compounds of the formula (IV) below).

| | Compound of | Compound of | Compound of |
|---|---|---|---|
| | the formula | the formula | the formula |
| Example | (II) | (III) or (IV) | (I) |
| 2 | 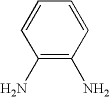 | DEC | 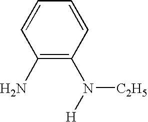 |
| 3 | 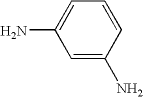 | DMC | 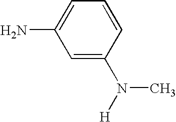 |
| 4 | 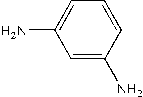 | DEC | 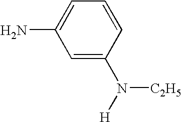 |
| 5 | 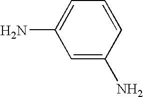 | EC | 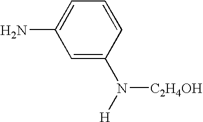 |
| 6 | 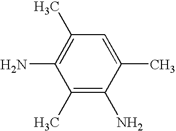 | DMC | 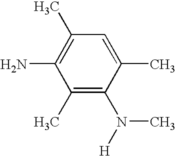 |
| 7 | 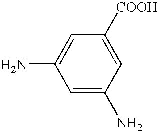 | DMC | 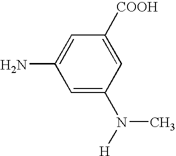 |
| 8 | 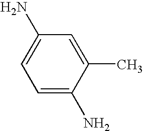 | DMC | 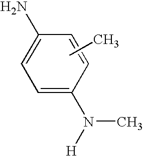 |

1-6. (canceled)

7. A process for preparing a compound of the formula (I) 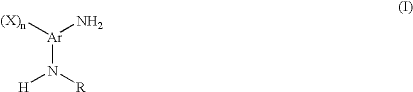
in which
Ar is an aromatic or heteroaromatic ring system;
X is a substituent;
n is from 0 to 6; and
R is an alkyl, hydroxyalkyl or a benzyl group;
which comprises reacting a compound of the formula (II) 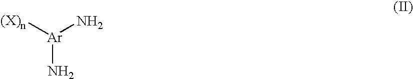
in which Ar, X and n are each as defined above with a compound of the formula (III)
(RO)₂Co (III)
in which R is as defined above or a compound of the formula (IV) 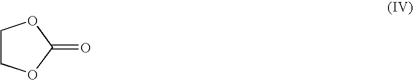
in the presence of a zeolite of the NaY faujasite type.

8. The process as claimed in claim 7, wherein
Ar is a benzene ring, a fused benzene ring, a pyridine ring or a pyrimidine ring;
X is C₁-C₄-alkyl, —COOR¹, —COR¹, —OR¹, —NHCOR¹, SO₂R², halogen, cyano, nitro, —SO₃M
or C₁-C₄-alkyl which is substituted by OR¹, OCOR¹ or by NHCOR¹,
in which R¹ is hydrogen, C₁-C₄-alkyl or C₁-C₄-alkyl which is substituted by OR¹, OCOR¹ or NHCOR¹;
R² is C₁-C₄-alkyl, halogen or C₁-C₄-alkyl which is substituted by OH or by OCOR¹; and
M is hydrogen or an alkali metal such as sodium, potassium or lithium;
n is 0, 1 or 2; and
R is C₁-C₄-alkyl, or hydroxy-C₁-C₄-alkyl;
where the —NH₂ group and the —NHR group may be ortho or meta to one another.

9. The process as claimed in 7, in which
Ar is a benzene ring;
X is methyl, ethyl, methoxy, ethoxy, —COOH, —COOMe, —COOEt, —NHCOMe, chlorine, cyano or nitro;
n is 0, 1 or 2; and
R is methyl, ethyl or hydroxyethyl.

10. The process as claimed in 8, in which
Ar is a benzene ring;
X is methyl, ethyl, methoxy, ethoxy, —COOH, —COOMe, —COOEt, —NHCOMe, chlorine, cyano or nitro;
n is 0, 1 or 2; and
R is methyl, ethyl or hydroxyethyl.

11. The process as claimed in claim 7, wherein the compound of the formula (I) has the formula (Ia)

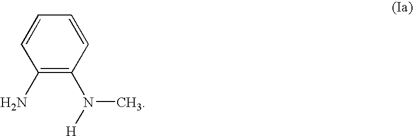

12. The process as claimed in claim 8, wherein the compound of the formula (I) has the formula (Ia)

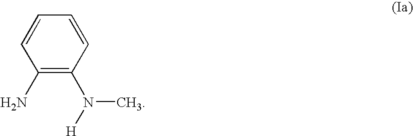

13. The process as claimed in claim 9, wherein the compound of the formula (I) has the formula (Ia)

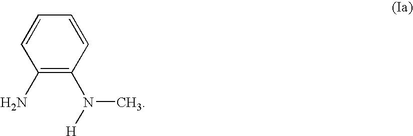

14. The process as claimed in claim 7, wherein the catalyst used is a modified zeolite of the NaY faujasite type.

15. The process as claimed in claim 7, wherein the modification is effected by addition of a lithium salt cocatalyst.

16. The process as claimed in claim 14, wherein the modification is effected by addition of a lithium bicarbonate or lithium carbonate.

17. The process as claimed in claim 16, wherein the modification is effected by addition of a lithium bicarbonate or lithium carbonate.
